(19) United States
(12) Patent Application Publication
(10) Pub. No.: US 2014/0107428 A1
(43) Pub. Date: Apr. 17, 2014

(54) IRRIGATING AND ILLUMINATING SURGICAL SYSTEM AND RELATED METHODS

(71) Applicant: Synergetics, Inc., O'Fallon, MO (US)

(72) Inventor: Matthew LaConte, Wildwood, MO (US)

(21) Appl. No.: 14/053,121

(22) Filed: Oct. 14, 2013

Related U.S. Application Data
(60) Provisional application No. 61/713,137, filed on Oct. 12, 2012.

(51) Int. Cl. A61B 3/117; A61M 3/02

(52) U.S. Cl. CPC A61B 3/117; A61M 3/0279
USPC 600/249; 29/428

(57) ABSTRACT

An illuminated surgical device includes a shaft and an optical fiber. The shaft includes a body having a central bore defined therein, and a passage extending through the shaft body to the central bore of the shaft. The shaft is configured for at least partial insertion into a human body. The optical fiber extends at least partially through the bore of the shaft such that light emitted from the optical fiber passes through the passage and is directed radially outward from the shaft.

### CROSS REFERENCE TO RELATED APPLICATIONS

This application claims the benefit of U.S. Provisional Application No. 61/713,137, filed Oct. 12, 2012.

### FIELD

This disclosure generally relates to ophthalmic surgical devices and, more specifically, to a system for illuminating and irrigating the anterior chamber of an eye, and to related methods.

### BACKGROUND

Some known surgical instruments are inserted through the cornea of the eye during certain eye procedures such as anterior segment surgery. During such procedures, pressure within the anterior chamber must be maintained. Typically, an anterior chamber maintainer including an infusion cannula is inserted through a corneal side port to provide irrigation to the anterior chamber and thereby maintain pressure in the chamber.

An illumination system may also be utilized during some procedures. However, conventional illumination systems may require a surgeon to hold the system in place. Also, the illumination systems may inadvertently direct light into the surgeon's eyes, thus hindering the surgical procedure. Accordingly, an improved anterior chamber maintainer system is needed.

This Background section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### BRIEF SUMMARY

In one embodiment, an illuminated surgical device is described. The device includes a shaft and an optical fiber. The shaft includes a body having a central bore defined therein, and a passage extending through the shaft body to the central bore of the shaft. The shaft is configured for at least partial insertion into a human body. The optical fiber extends at least partially through the bore of the shaft such that light emitted from the optical fiber passes through the passage and is directed radially outward from the shaft.

In another embodiment, an illuminated surgical system is described. The system includes a connector, a shaft, and an optical fiber. The shaft is coupled to the connector, and includes a body having a central bore defined therein and a passage extending through the shaft body to the bore of the shaft. The shaft is configured for at least partial insertion into a human body. The optical fiber is coupled to the connector, and extends at least partially through the bore of the shaft along the passage. The shaft is configured to direct light from the optical fiber radially outward through the passage.

In yet another embodiment, a method of fabricating an illuminated surgical system is described. The method includes fabricating a hollow shaft having a central bore defined therein, forming a passage through a wall of the shaft, the passage extending through the wall to the central bore, and inserting an optical fiber at least partially through the bore of the shaft such that light emitted from the optical fiber passes through the passage and is directed radially outward from the shaft.

Various refinements exist of the features noted in relation to the above-mentioned aspects. Further features may also be incorporated in the above-mentioned aspects as well. These refinements and additional features may exist individually or in any combination. For instance, various features discussed below in relation to any of the illustrated embodiments may be incorporated into any of the above-described aspects, alone or in any combination.

### BRIEF DESCRIPTION OF THE DRAWINGS

FIG. 1 is a schematic view of an embodiment of a surgical system;

FIG. 2 is an enlarged view of the system of FIG. 1;

FIG. 3 is a cross-sectional view of the system and taken along line 3-3 of FIG. 2;

FIG. 4 is an exploded view of the system; and

FIG. 5 is a schematic view of the system positioned within an eye of the body during surgery.

### DETAILED DESCRIPTION

This disclosure generally relates to surgical systems that provide radially directed light and irrigation fluid to the surgical site through a common passage. The system generally includes structure to bridge the anterior chamber of the eye, to maintain the shaft in the proper or selected orientation, and to deliver illumination and irrigation fluid to a selected location. As further described below, a surgeon can select an orientation for the system, and the system automatically maintains the selected orientation until the system is moved or removed. In the described embodiment, the system also enables maintenance of the anterior chamber such that it is not damaged during surgery. The system maintains the correct or selected orientation in the body, without requiring the surgeon to hold the system in place. Systems consistent with this disclosure may be used in various surgical procedures such as pediatric ophthalmic procedures, and in particular, intraocular manipulations or treatment during anterior segment surgery. While the system described herein is configured for ophthalmic procedures, systems consistent with this disclosure may also be adapted for and used in other surgical procedures.

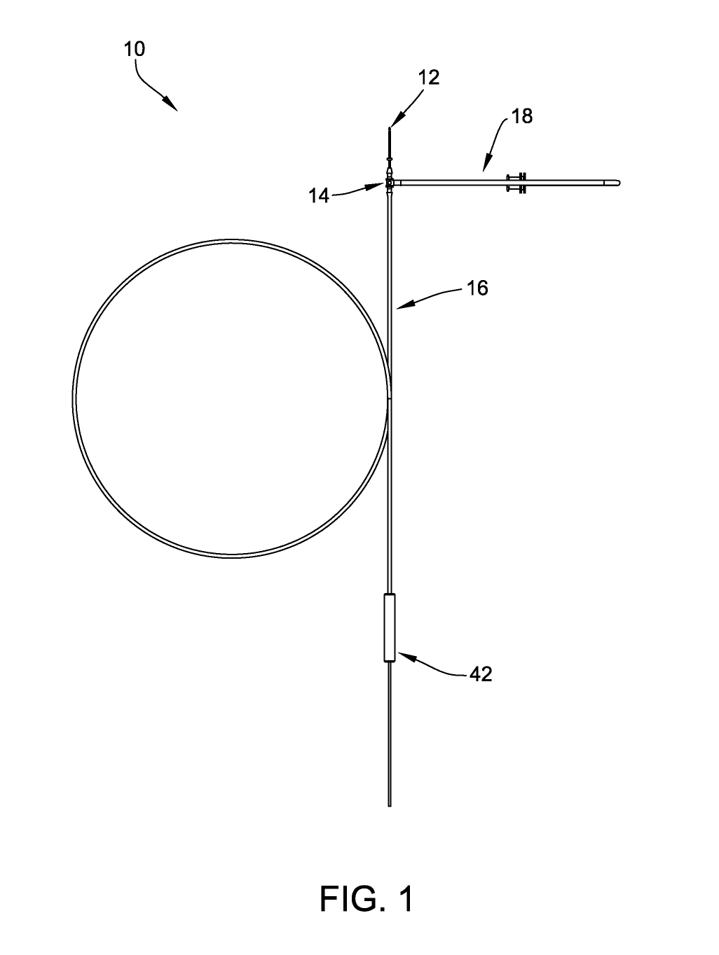

Referring to FIG. 1, an illuminated surgical system of one embodiment is generally indicated by reference numeral 10. System 10 includes a shaft 12, a connector 14, a fiber optic cable 16 and an infusion line 18. System 10 of this embodiment is configured for use in anterior segment surgery, and more specifically for providing irrigation (maintaining the anterior chamber) and for illumination in the anterior chamber. In this embodiment, the system provides radial illumination to facilitate the procedure and to reduce the risk of light being directed toward the surgeon. Alternatively, the system 10 may be used for other surgical procedures.

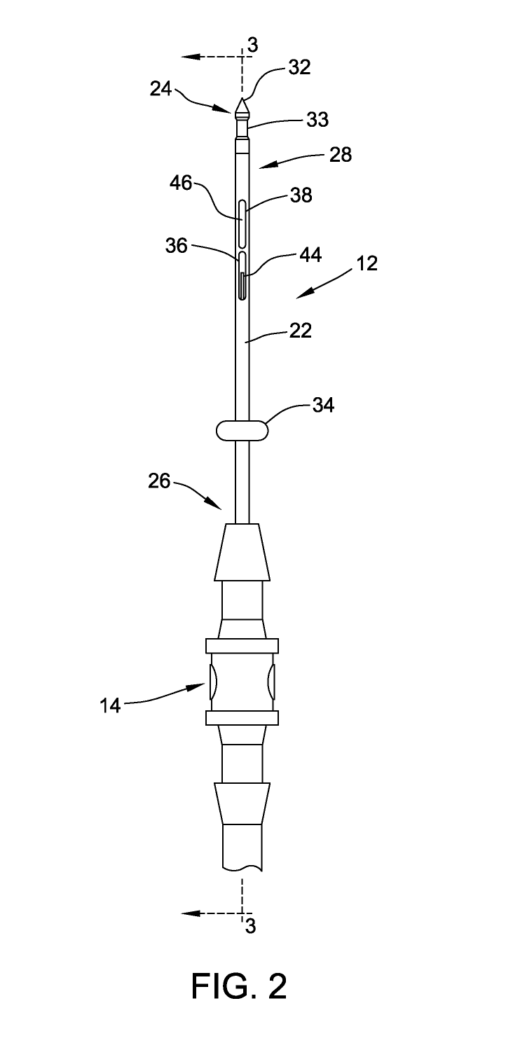

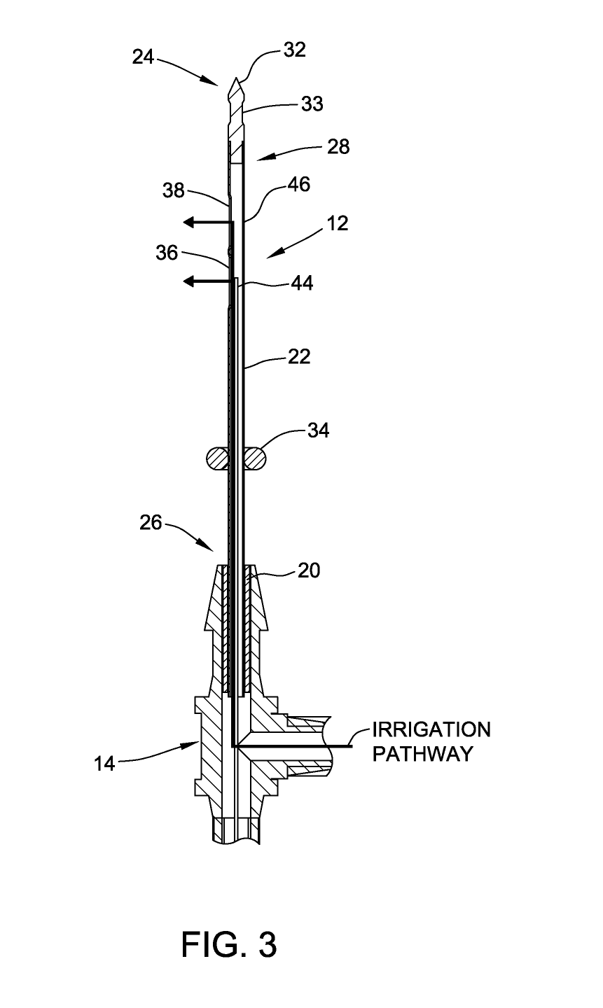

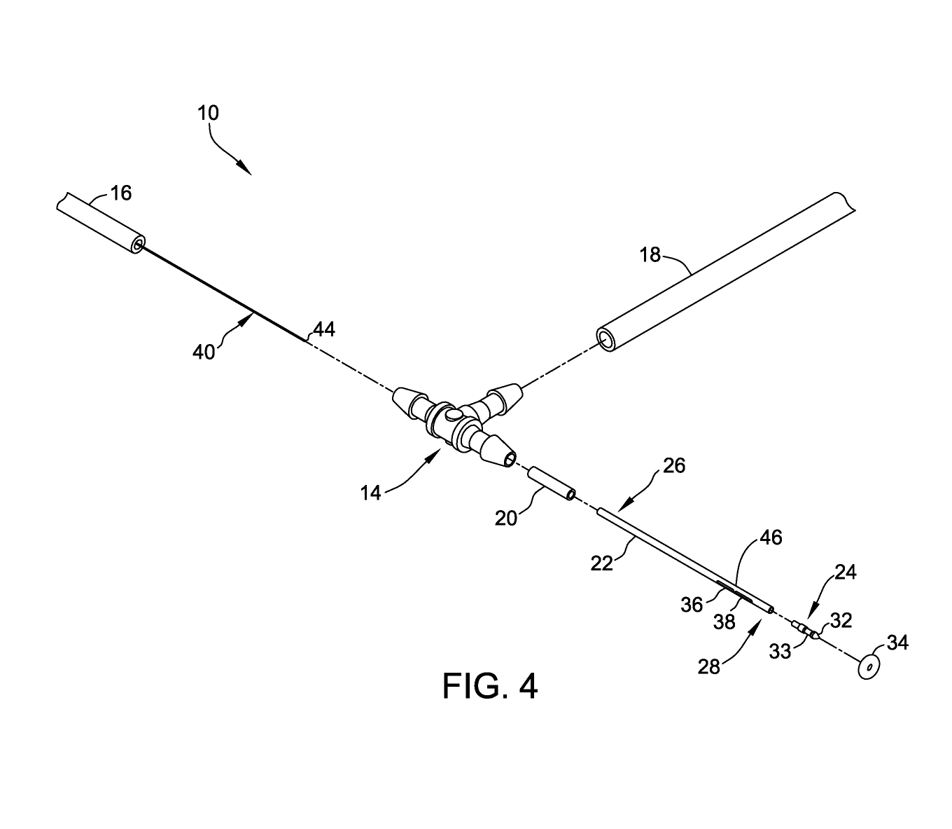

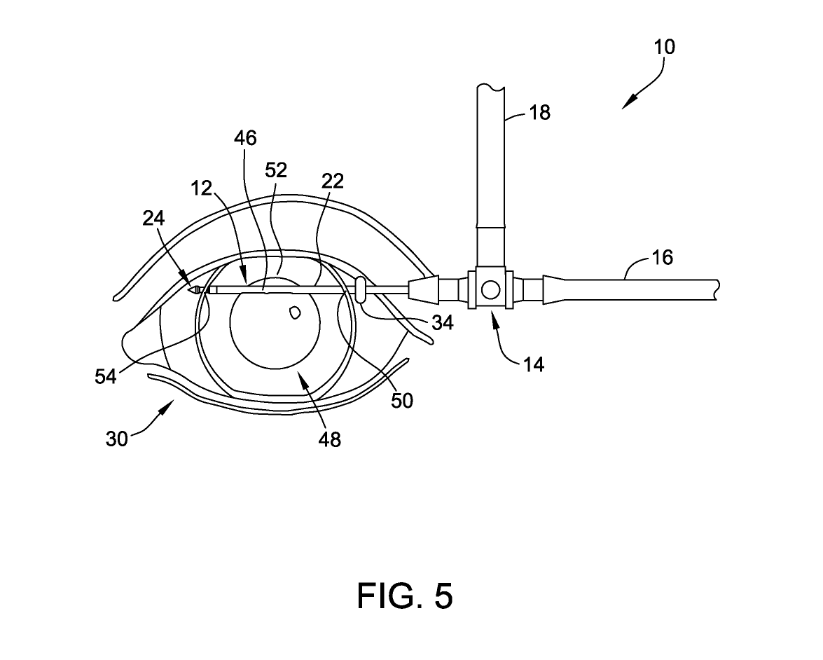

Referring to FIGS. 2-4, shaft 12 includes a base 20 (FIGS. 3-4), a tubular body 22, and a head 24. Base 20 is coupled to connector 14 and a first end 26 of body 22 is coupled to base 20. Head 24 is coupled to a second end 28 of body 22. Shaft 12 is insertable into a body such as an eye 30 of a human body (FIG. 5). Head 24 includes a conical tip 32 to facilitate insertion into the body and a recessed portion 33 to facilitate maintaining shaft 12 in a selected orientation within the human body. In the illustrated embodiment, head 24 is shown as a separate, distinct element, although it is contemplated that head 24 may be integrally formed with body 22. A stop 34 is slidably coupled to first end 26 for positioning against the body (e.g., an eye) to facilitate maintaining shaft 12 in a selected orientation relative to the body.

As described above, shaft 12 has a tubular body 22. As such, shaft 12 has a central bore extending longitudinally through shaft 12. Shaft 12 further includes a first passage 36 and a second passage 38 formed through the wall of body 22. Although shaft 12 is illustrated as having two passages, shaft 12 may have any number of passages (e.g., one, three, etc.). In this embodiment, passages 36 and 38 are sized and spaced to maintain a rigid shaft 12 for insertion while maximizing irrigation flow output. In embodiments having more or less than two passages, the passages can be suitably sized and spaced to maintain the rigidity of shaft 12 for insertion while maximizing irrigation flow output. For example, one suitable embodiment includes three axially aligned passages each having a size smaller than passages 36 and 38 to maintain the rigidity of shaft 12. Further, the depth of passages 36 and 38 (i.e., the distance passages 36 and 38 extend into body 22 of shaft 12) is suitably less than 50 percent of the diameter of shaft 12 to maintain the rigidity of shaft 12.

Passages 36 and 38 are axially aligned on shaft body 22 so that the passages are centrally located above the pupil of the eye after insertion. However, passages 36 and 38 may have different orientations relative to one another, but should generally be centrally located above the pupil of the eye after insertion. Passages 36 and 38 facilitate directing fluid and/or light into the body during a surgical procedure, as is described herein.

Fiber optic cable 16 is coupled to connector 14 and includes an optical fiber 40 connected to a light-source connector 42 (e.g., a multiport adapter) (FIG. 1), which is connected to a light source (not shown). Optical fiber 40 extends from cable 16 through connector 14 and into shaft 12. Optical fiber 40 includes a first end 44 positioned within shaft body 22 and may be positioned along first passage 36 and/or second passage 38 such that light transmitted through optical fiber 40 is directed radially outward through passage 36 and/or 38 from shaft body 22. Shaft body 22 includes a solid inner wall 46 positioned opposite passages 36 and 38 to act as a light reflector to facilitate directing light outward from passage 36 and/or 38 and to act as a shield such that light is not permitted to exit radially from shaft body 22 along some portion of its circumference. Additionally, head 24 may prevent light from exiting axially from shaft body 22. As such, light is directed radially from only a portion of the circumference of shaft body 22 (e.g., through passages 36 and/or 38) and can be directed in a selected direction.

In the example embodiment, infusion line 18 is coupled to connector 14. Infusion line 18 is coupled to a fluid source (not shown) to deliver irrigation fluid into shaft 12 and out through first passage 36 and/or second passage 38. The space between shaft body 22 and optical fiber 44 is sufficiently wide to allow adequate flow of irrigation fluid to the body (e.g., an eye) for the selected surgical procedure.

Referring to FIG. 5, illuminated surgical system 10 may be used to perform intraocular manipulations during surgery involving the cornea 48 of an eye 30. In use, shaft head 24 is inserted through a first corneal side port 50, through an anterior chamber 52, and through a second corneal side port 54. The cornea of second corneal side port 54 is retained within recess portion 33 (FIGS. 2-4), which facilitates preventing axial movement of shaft 12. Stop 34 is moved along shaft body 22 towards first corneal side port 50 until it rests against the cornea wall to further facilitate preventing axial movement of shaft 12. As such, shaft 12 is maintained across anterior chamber 52. Irrigation fluid is delivered through infusion line 18 and through first passage 36 and/or second passage 38 to prevent inadvertent collapse of anterior chamber 52 during intraocular manipulation. Light travels from a light source through optical fiber 40 to fiber first end 44 located within shaft body 22. Light emanating from optical fiber 40 passes through first passage 36 and/or second passage 38 to illuminate anterior chamber 52 and the surrounding area. The surrounding area may include the posterior segment of the eye which may be illuminated through the pupil. Since light is directed radially from only a portion (e.g. through passage 36 and/or 38) of shaft body 22, a surgeon may rotate shaft 12 to direct light in a different direction. In this way, inner wall 46 may be faced away from the surgeon so light from optical fiber 40 does not shine into the surgeon's eyes and disrupt the surgical procedure.

When introducing elements of the present invention or the embodiments thereof, the articles “a”, “an”, “the” and “said” are intended to mean that there are one or more of the elements. The terms “comprising”, “including” and “having” are intended to be inclusive and mean that there may be additional elements other than the listed elements. The use of terms indicating a particular orientation (e.g., “top”, “bottom”, “side”, etc.) is for convenience of description and does not require any particular orientation of the item described.

As various changes could be made in the above constructions and methods without departing from the scope of the invention, it is intended that all matter contained in the above description and shown in the accompanying figures shall be interpreted as illustrative and not in a limiting sense.

What is claimed is:

1. An illuminated surgical device comprising:
a shaft including a body having a central bore defined therein and a passage extending through the shaft body to the central bore of the shaft, the shaft configured for at least partial insertion into a human body; and
an optical fiber extending at least partially through the bore of the shaft such that light emitted from the optical fiber passes through the passage and is directed radially outward from the shaft.

2. The device of claim 1, further comprising a head coupled to the shaft, the head including a recessed portion to maintain a position of the shaft within the human body.

3. The device of claim 1, further comprising a stop slidably coupled to the shaft, the stop configured to position against the human body to maintain a position of the shaft within the body.

4. The device of claim 1, wherein the shaft comprises a shielding portion opposite the passage, the shielding portion configured to direct light through the passage.

5. The device of claim 1, wherein the passage comprises a first passage and a second passage.

6. The device of claim 5, wherein light is directed radially outward from the first passage, and at least one of the first and second passages are configured to deliver an irrigation fluid into the body.

7. The device of claim 1, wherein the shaft is configured to be inserted into a cornea of an eye and retained by the eye such that the shaft is positioned across an anterior chamber of the eye for illumination thereof.

8. An illuminated surgical system comprising:
a connector;
a shaft coupled to the connector, the shaft including a body having a central bore defined therein and a passage extending through the shaft body to the bore of the shaft, the shaft configured for at least partial insertion into a human body; and
an optical fiber coupled to the connector, the optical fiber extending at least partially through the bore of the shaft along the passage, wherein the shaft is configured to direct light from the optical fiber radially outward through the passage.

9. The system of claim 8, further comprising a head coupled to the shaft, the head including a recessed portion to maintain a position of the shaft within the human body.

10. The system of claim 8, further comprising a stop slidably coupled to the shaft, the stop configured to position against the human body to maintain a position of the shaft within the human body.

11. The system of claim 8, wherein the shaft comprises a shielding portion opposite the passage, the shielding portion configured to direct light through the passage.

12. The system of claim 8, wherein the passage comprises a first passage and a second passage, and at least one of the first and second passages are configured to deliver irrigation fluid into the human body.

13. The system of claim 8, further comprising an infusion line coupled to the connector, the infusion line configured to direct an irrigation fluid to the shaft and through the passage.

14. The system of claim 8, wherein the shaft is configured to be inserted into a cornea of an eye and retained by the eye such that the shaft is positioned across an anterior chamber of the eye for illumination thereof.

15. A method of fabricating an illuminated surgical system, the method comprising:
fabricating a hollow shaft having a central bore defined therein;
forming a passage through a wall of the shaft, the passage extending through the wall to the central bore; and
inserting an optical fiber at least partially through the bore of the shaft such that light emitted from the optical fiber passes through the passage and is directed radially outward from the shaft.

16. The method of claim 15, further comprising coupling the shaft to a connector; and
coupling an infusion line to the connector, the infusion line configured to direct an irrigation fluid to the shaft and through the passage.

17. The method of claim 16, wherein forming a passage comprises forming a first passage and forming a second passage through a wall of the shaft, the first passage configured to deliver an irrigation fluid into a human body, the second passage configured to direct light emitted from the optical fiber radially outward from the shaft.

18. The method of claim 15, further comprising slidably coupling a stop to the shaft, the stop configured to position against a human body to maintain a position of the shaft within the human body.

19. The method of claim 15, wherein forming a passage comprises forming a first passage and forming a second passage through the wall of the shaft.

20. The method of claim 15, further comprising forming a shielding portion opposite the passage, the shielding portion configured to direct light through the passage.
